(19) United States
(12) Patent Application Publication
(10) Pub. No.: US 2015/0290025 A1
(43) Pub. Date: Oct. 15, 2015

(54) Oral System

(71) Applicant: Federico Valdemira, Lake Ronkonkoma, NY (US)

(72) Inventor: Federico Valdemira, Lake Ronkonkoma, NY (US)

(21) Appl. No.: 14/248,952

(22) Filed: Apr. 9, 2014

(51) Int. Cl. A61F 5/56; A61C 5/14

(52) U.S. Cl. CPC A61F 5/566; A61C 5/14; A61F 2005/563

(57) ABSTRACT

An attachment system for use with an intra-oral dental appliance to be inserted into the oral cavity of a human for treatment of sleep apnea, snoring, bruxism, and temporo-mandibular joint disorder, the attachment system having: a first attachment means securable to a bilateral under surface conforming to the clinical occlusal plane of a human, and to the maxillary dentition of a first molded member; a second attachment means securable to a bilateral upper surface conforming to the clinical occlusal plane of a human, and to the mandibular dentition of a second molded member; an attachment element having means for connecting the first attachment means, and connecting the second attachment means cooperatively together.

### CROSS REFERENCE TO RELATED APPLICATIONS

None

### BACKGROUND OF THE INVENTION

The present invention provides a non-surgical oral system, including, inter alia, a system that enables an individual to sleep with mandibular (i.e., lower jaw) repositioning. More particularly, the present invention provides a custom fitted mandibular repositioning system that is readily customized while reducing and optimally eliminating tissue impingement having attachment situated between the mandible and maxillary (i.e., upper jaw) bite block or occlusal (i.e., biting surface of the molars) planes permitting structural stability and integrity. Moreover, the system provides for infinite horizontal adjustability within the dimensions of the system.

The invention provides treatment of sleep apnea, snoring, bruxism (i.e., grinding of the teeth), and other now known or future diagnosed oral, respiratory, dental and developmental disorders that may affect the dentition and surrounding skeletal and muscular structures. In particular, the present invention provides an improved system for enhanced precision and control of interocclusal devices used in patient therapy.

Difficulty in breathing while sleeping often produces snoring or the more extreme obstructive sleep apnea. Snoring is a condition affecting approximately forty percent (40%) of the adult population, while sleep apnea is believed to affect at least twenty (20) million people.

Bruxism consists of a static and/or dynamic contact between the dentition of the mandible and maxilla; and is commonly referred to as “tooth grinding” which yields physical destruction of tooth enamel directly leading to tooth decay in addition to more serious issues, and affects nearly fifteen percent (15%) of the population, whereas in advanced stages bruxism yields the abnormal and excessive grinding or clenching of teeth while an individual is fast asleep.

Medically, bruxism is classified as parasomnia or a sleep disorder, in fact, in some instances a person may be so unaware of the problem that it may even occur when one is awake.

Although the actual cause of bruxism remains a mystery, contributing factors appear to be related, inter alia, to emotional stress or other psychological factors.

Common treatments range from psychotherapy, sedatives or tranquilizers and may further include bio-feedback, i.e. electrodes being taped to the jaw.

Temporomandibular joint syndrome (or dysfunction) (or more commonly know as “TMJ”) is a disorder effecting the joint between the mandible and the skull, wherein the temporomandibular joint lies between the temporal bone of the skull and the mandible of the jaw, and allows the jaw to open and close. The joint is formed by a condyle on the mandible which hinges and glides in and out of the depression in the temporal bone.

TMJ can be caused by bruxism, malocclusion, trauma, and arthritis, however, there is also an indication that a posterior or backward displacement of the condyle of the jaw significantly contributes to TMJ pain. TMJ has been associated with a wide variety of physical aliments, including migraine headaches which can become so extreme thereby yielding nausea and blurred vision. Most people afflicted with TMJ suffer from a myo-facial pain-dysfunction syndrome primarily as a muscle problem related to dental/skeletal relationships and tensional factors. The effects can range from mild to severe, including pain in the joint area that can extend to the shoulders, back, neck, and sinuses; whereas surgery is typically reserved for the most severe cases, which represent about ten percent (10%) of those seeking treatment.

Accordingly, the medical profession and related health care industries have recently begun to recognize the long felt need of developing new and innovative techniques that effectively address the aforementioned plethora of medical issues. Hence, oral appliances (e.g., interocclusal devices) have been employed to prevent the tongue and/or collapse of oral pharyngeal tissue from obstructing the airway. To date, when used in the treatment of snoring or sleep apnea, these prior techniques and devices have been fallen short in results, e.g., the prior approaches have been underperforming, and/or caused undesirable side effect(s).

As such, there continues to be a need for providing a system that maintains the mandible in a preferred protruded position, while simultaneously permitting a limited degree of lateral and protrusive movement thereby minimizing the negative effects of a static positioning of teeth and the related muscles and ligaments; temporomandibular joint irritation or aggravation; impingement or disruption of tongue airway/architecture and flow, individually and in combination; and rigid fixation of teeth to limit tooth movement and undesirable occlusal change.

Moreover, there is an additional need to overcome the deficiencies of the industry, wherein there is a risk that the upper and lower subassemblies become unhinged or separated apart during use, this is especially dangerous, and possibly fatal if swallowed when the patient/user is fast asleep. It is this issue and more that the present invention is designed to overcome.

### SUMMARY OF THE INVENTION

Methods and devices that allow for articulation and disarticulation and positioning of the mandible in relation to the maxilla are disclosed. Such movement and ranges of motion coupled with the unique and novel manners of attachment between the upper and lower relational members allow precise control of mandibular placement. Moreover, the precise positioning allows for a range of motion in static and functional use. Furthermore, the ability to articulate and disarticulate allows for comfort in insertion and application in the patient's mouth and allowing for divergent paths of insertion and withdrawal between the invention's means for articulation. The device provides for infinite adjustability within the dimensions of the oral cavity, whilst ensuring that the system remains interlocked when deployed in the patient's/user's mouth during use thereof.

An attachment system for use with an intra-oral dental appliance to be inserted into the oral cavity of a human for treatment of sleep apnea, snoring, bruxism, and temporo-mandibular joint disorder, said attachment system comprising: a first attachment means (e.g., top apparatus) securable to a bilateral under surface conforming to the clinical occlusal plane of a human, and to the maxillary dentition of a first molded member; a second attachment means (e.g., bottom apparatus) securable to a bilateral upper surface conforming to the clinical occlusal plane of a human, and to the mandibular dentition of a second molded member; an attachment element (e.g., screw & nut) comprising means for connecting said first attachment means, and connecting said second attachment means cooperatively together.

An attachment system wherein said first attachment means comprises a body having a oval shape with a top and bottom and an elongated opening in the middle for said attachment element to adjustably pass there through; wherein said top includes a flange extending inwardly, and has adjustment indicators for use with said attachment element; wherein said base includes a flange extending outwardly for securing the first attachment means to the first molded member; said body having a wall between the top and bottom and runs from the length of a first long side, around a first curve (rear of mouth) to a second long side running the length thereof forming a chamber.

An attachment system wherein said second attachment means comprises a body having an oval shape with a top and bottom, and with an elongated opening in the middle having a proximal and distal end said opening defined by a rim having a length, width, and height, a topside, and bottom side extending inwardly from the top of said body providing for said attachment element to adjustably pass there through; a first region of said rim having a length, width, and height being substantially elliptical in shape beginning near the proximal end (front of the mouth) of said top of said body; a second region positioned near said distal end (rear of the mouth) of said rim, is substantially circular, and intersects said first region; and wherein a section of the topside of the rim comprising a positioning means for receiving said attachment element, said rim having a varying height forming a declined plane beginning near the proximal end and terminating at the intersection of the second region.

An attachment system wherein said attachment element (e.g., screw & nut) comprises a male and a female member; said male member having a head, a neck, a waist, and a tail portion; said head and waist portion each having a diameter both of which are larger than the diameter of said neck and tail portions; the head further having a height, a top, a bottom, and a side, wherein the top has a diameter less than the bottom, and a rounded edge (0.026 inch rad) connecting the top and side surfaces of the head; said head portion being configured and dimensioned to pass through said second region but not said first region, wherein said head can only pass through said second region at an of angle of greater than eight (8) degrees+/−3 degrees; said neck portion configured and dimensioned to pass through both regions, and to slide freely between said proximal and distal end within both regions; said waist portion being configured and dimensioned to pass through said second region, but not said first region; said tail portion having a height, and diameter, with a threaded means about the sides configured and dimensioned to mate with said female member; said female member comprising a body having a diameter, height, top, bottom, and sides, and orifice in the center of said body configured and dimensioned to mate with said tail portion; the sides defining an outer perimeter wherein the front, and rear sides each have a radius, wherein the rear perimeter has a radius greater than the front, wherein the front radius is 0.020 inches, and rear radius is 0.079 inches, wherein the orientation has the larger radius facing the rear of the mouth because the curved portion is facing towards the rear of the mouth. The rear portion begin configured to mate with the curved end of the chamber of said first attachment means; and the sides being configured to slide within the chamber but prohibit rotational movement of said female member within the chamber; wherein said diameter is greater than the narrow portion of the elongated opening of the first attachment means.

Many other advantages and features of the present invention will become obvious to those familiar in the art with review and reference to the detailed description and accompanying drawings.

Thus, it is an object of the present invention to provide a interocclusal repositioning appliance that may be is custom or mass produced and therein fitted and easily adjustable for the treatment of sleep disordered breathing, bruxism etc., and other above mentioned maladies and afflictions.

Another object of the invention is to provide for a protrusive infinitely adjustable oral appliance within the dimensions of the oral cavity.

Another object of the invention is to provide a device of the type described which does not cause discomfort to the user or harm to the teeth, tongue, temporomandibular joint or gums during use.

Another object of the invention to provide an improved oral cavity device that can be used by persons to give relief from snoring and sleep apnea, and that provides for breathing through the mouth, nares or both.

Still another object of the invention is to provide a device of the type described which is of durable and reliable construction since therapy may be required for life.

Yet another object of the invention is to avoid and minimize alteration of the oral airways architecture.

Another object of the invention is to provide a device that minimizes, alleviates or eliminates tongue irritation or contact with an oral appliance or parts thereof.

Another object of the invention is to reduce or eliminate the impingement upon tongue space.

Another object of the invention is to aid in the treatment of TMJ disorders.

Still another object of the invention is to reduce or eliminate bruxism and its negative effects.

Another object of the invention is to permit for a range of protrusive motion to aid in swallowing.

Another object of the invention is to permit for a range of lateral motion.

Another object of the invention is to reduce or eliminate the impingement upon vestibular space.

Another object of the invention is to allow for precision placement of the mandible in relation to the maxilla.

Still further, it is an object of the invention to provide a system that is infinitely adjustable while remaining safely engaged together.

An additional object of the invention is provide a positive locking mechanism wherein the system remains engaged when deployed in the mouth of the patient.

A further objective of the invention provides a device for individuals that suffer from sleep apnea.

An additional objective of the invention provides a device for individuals that travel, so that they need not cart a CPAP machine about, and can readily tote a small cup for the appliance.

A still additional objective of the invention, is to provide a device of individuals that are claustrophobic and who can not use CPAP machines.

Another object of the invention is providing a device that can be used as an Orthodontic Appliance to maintain lower teeth position.

Other objectives, advantages, and novel features of the present invention will become more apparent from the following detailed description when taken in conjunction with the accompanying drawings.

### BRIEF DESCRIPTION OF THE DRAWINGS

The following drawings, in which like reference characters indicate like parts, are provided for illustration of the invention and are not intended to limit the invention in any manner whatsoever.

FIG. 1A illustrates a bottom view of the first attachment means;

FIG. 1B illustrates a front view of the first attachment means;

FIG. 1C illustrates a left end view of the first attachment means which is orientated towards front of the mouth;

FIG. 1D illustrates a right end view of the first attachment means which is orientated towards to rear of the mouth;

FIG. 1E illustrates a top view of the first attachment means;

FIG. 1F illustrates an angled view of the first attachment means;

FIG. 2A illustrates a side view of the screw portion of an attachment element;

FIG. 2B illustrates a top view of the screw portion of an attachment element;

FIG. 2C illustrates a side view of the nut portion of an attachment element;

FIG. 2D illustrates a top view of the nut portion of an attachment element;

FIG. 3A illustrates a bottom view of the second attachment means;

FIG. 3B illustrates a front view of the second attachment means;

FIG. 3C illustrates a left end view of the second attachment means;

FIG. 3D illustrates a sectional view of the right end of the second attachment means along lines A-A;

FIG. 3E illustrates a top view of the second attachment means;

FIG. 3F illustrates an angled view of the second attachment means;

FIG. 3G illustrates a sectional view along lines B-B

FIG. 4A illustrates a side view of the attachment element installed on the first attachment means and how it is inserted into the second attachment means, thereby depicting the positive locking mechanism feature;

FIG. 4B illustrates a side view of the assembly when engaged and in use with molded members;

FIG. 4C illustrates a top view of the first attachment means positioned on a first molded member; and

FIG. 4D illustrates a top view of the second attachment means position on a second molded member.

### DETAILED DESCRIPTION OF INVENTION

All of the above referenced patents; patent applications and publications are hereby incorporated by reference. Many variations of the present invention will suggest themselves to those of ordinary skill in the art in light of the above detailed description. All such obvious modifications are within the full-intended spirit and scope of the claims of the present application, including equivalents thereof.

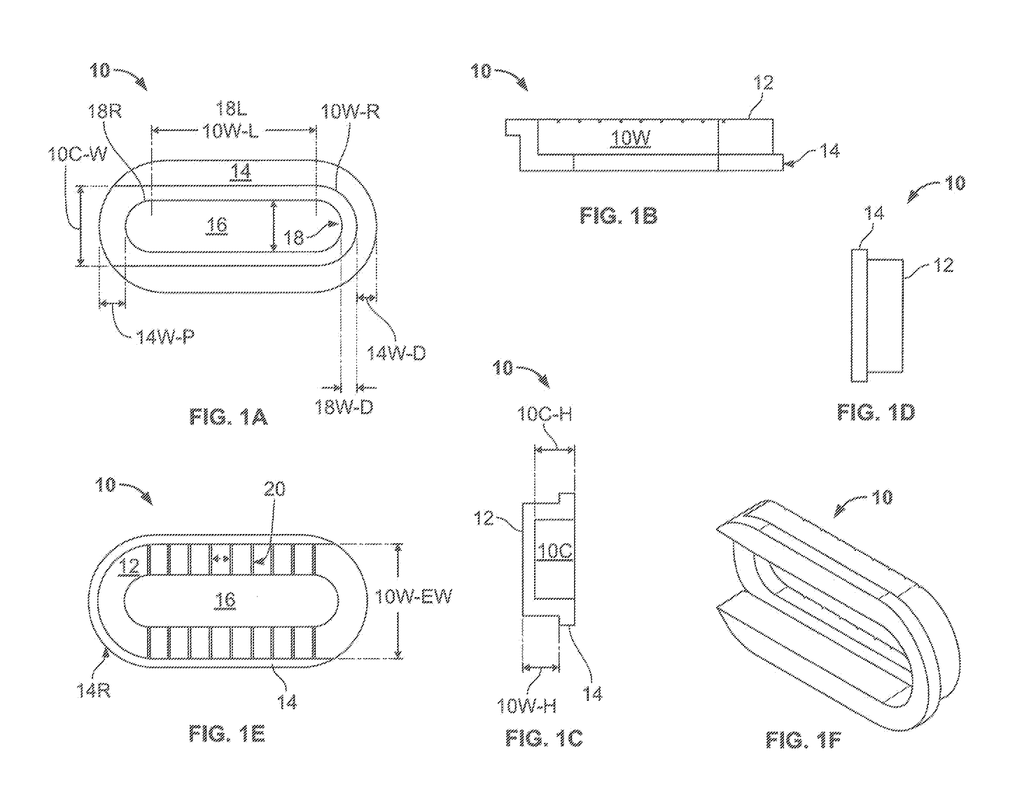

Referring generally to FIGS. 1A to 4D, hereinbelow, an attachment system 1 for use with an intra-oral dental appliance DA to be inserted into the oral cavity of a human for treatment of sleep apnea, snoring, bruxism, and temporo-mandibular joint disorder. The attachment system 1 includes a first attachment means 10 or top apparatus securable to a bilateral under surface conforming to the clinical occlusal plane of a human, and to the maxillary dentition of a first molded member MM1.

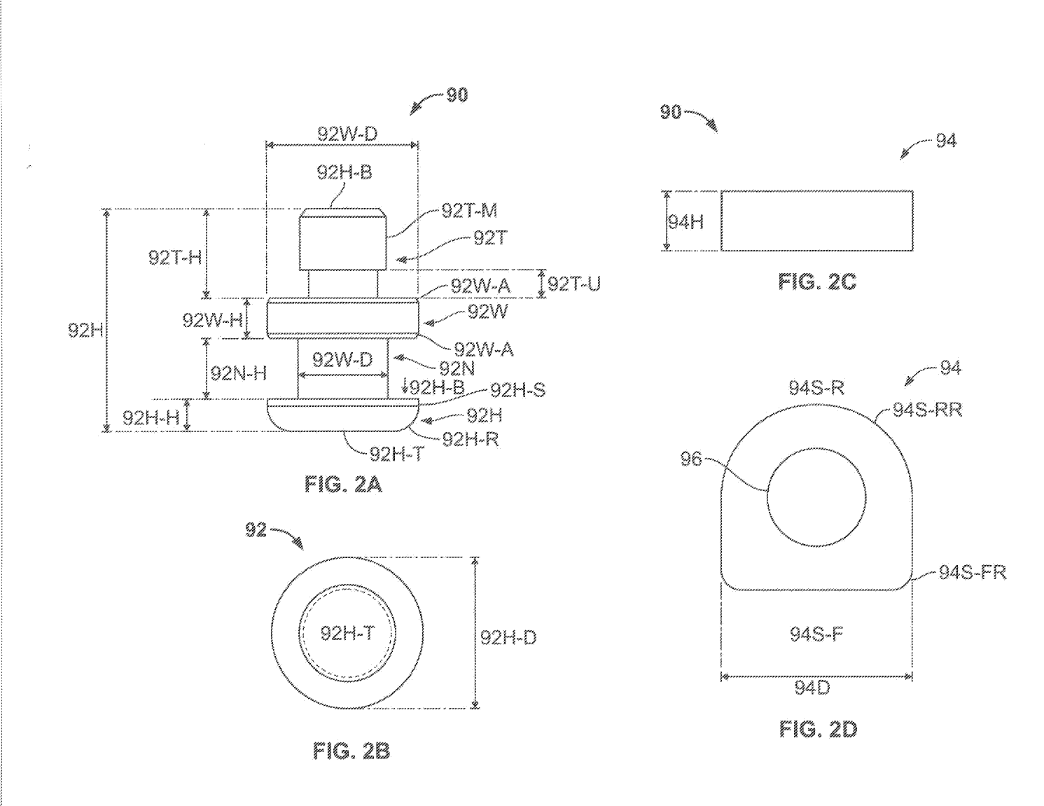

FIGS. 1A through 1F collectively illustrate the first attachment means 10, which includes a body having an oval shape with a top 12 and bottom 14 and an elongated opening 16 in the middle for said attachment element 90 to adjustably pass therethrough. The top 12 includes a flange 18 extending inwardly, defining the opening 16 which in the preferred embodiment is 0.102 inches wide at the narrowest point, and 0.422 at its widest point. Top 12 further includes adjustment indicators 20 for use with said attachment element 90 as illustrated collectively in FIGS. 2A to 2D, wherein the indicators 20 are spaced 0.040 inches apart.

The base 14 includes a flange extending outwardly for securing the first attachment means 10 to the first molded member MM1, while the body having a wall 10W between the top 12 and bottom 14 and runs from the length of a first long side, around a first curve (orientated towards the rear of mouth) to a second long side running the length thereof forming a chamber 10C. The distance between the opening 16 and the perimeter of the base 14 is 0.049 inches on the proximal end as shown by 14W-P; and on the first curved end or distal end, the distance of the flange between the 10W and the outer perimeter of bottom 14 is shown as 14W-D as 0.039 inches; and the distance of the flange 18 extending inwardly is 0.030 inches and shown as 18W-D. Furthermore, the radius of bottom 14 on the curved ends are 0.130 inches as denoted by 14R.

The length of the wall 10W on the first and second side is 0.320 inches, wherein the radius of the wall is 0.080 inches as shown by 10W-L and 10W-R respectively. The length of the flange 18 on the parallel sides is 0.320 each and the radius about the curved ends are 0.051 inches each as shown by 18R and 18L respectively. Moreover, in a preferred embodiment, chamber 10C has a width 10C-W of 0.160 inches, a height 10C-H of 0.070 inches, and the wall 10W has a height 10W-H of 0.070 inches. The wall 10W having an exterior width of 0.220 inches, as illustrated by 10W-EW.

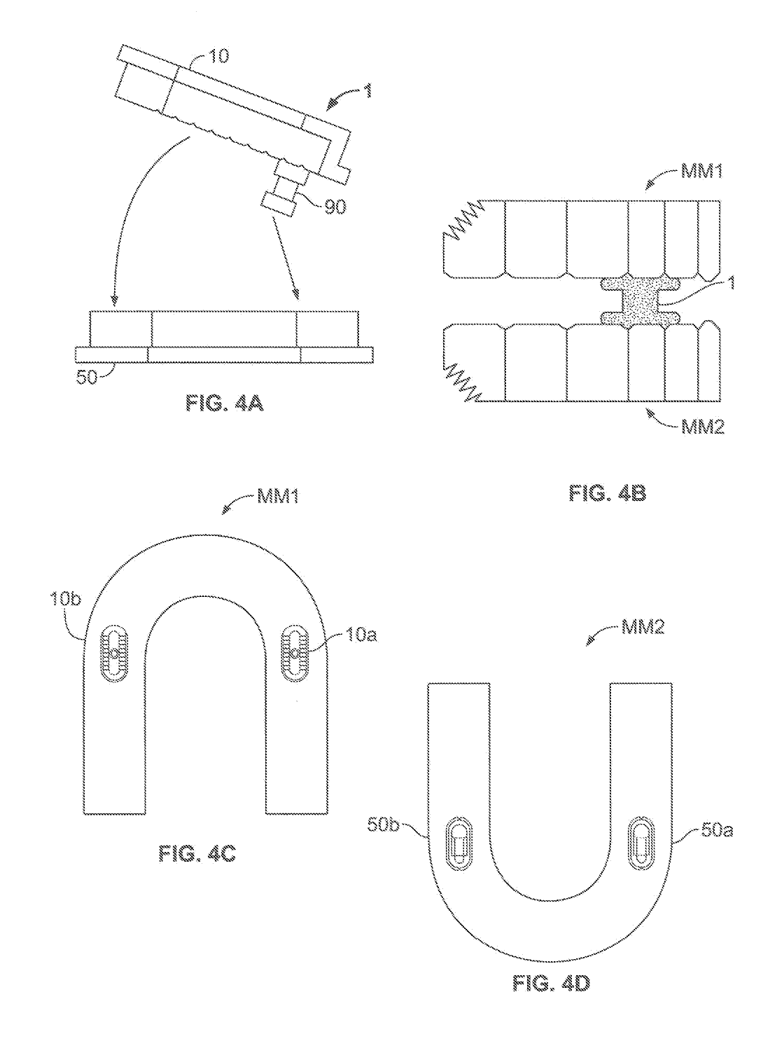

The system 1 further includes a second attachment means 50 (bottom apparatus) securable to a bilateral upper surface conforming to the clinical occlusal plane of a human, and to the mandibular dentition of a second molded member, MM2 as illustrated in use collectively in FIGS. 4A to 4D.

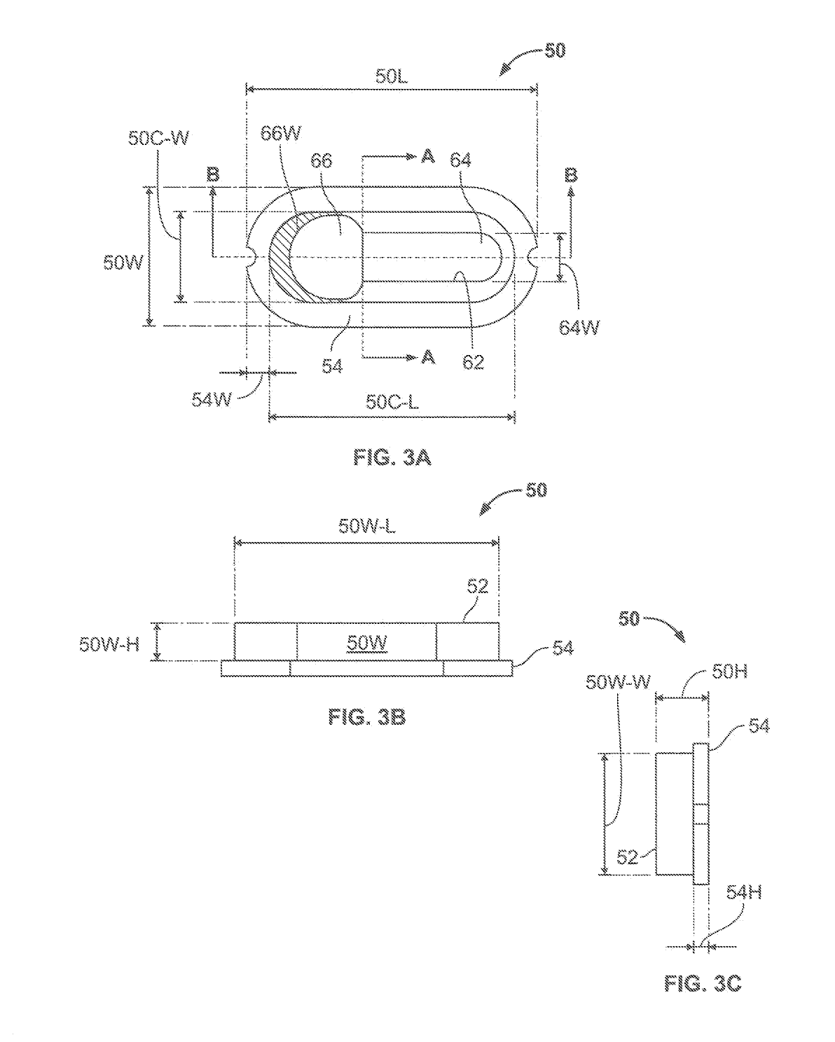

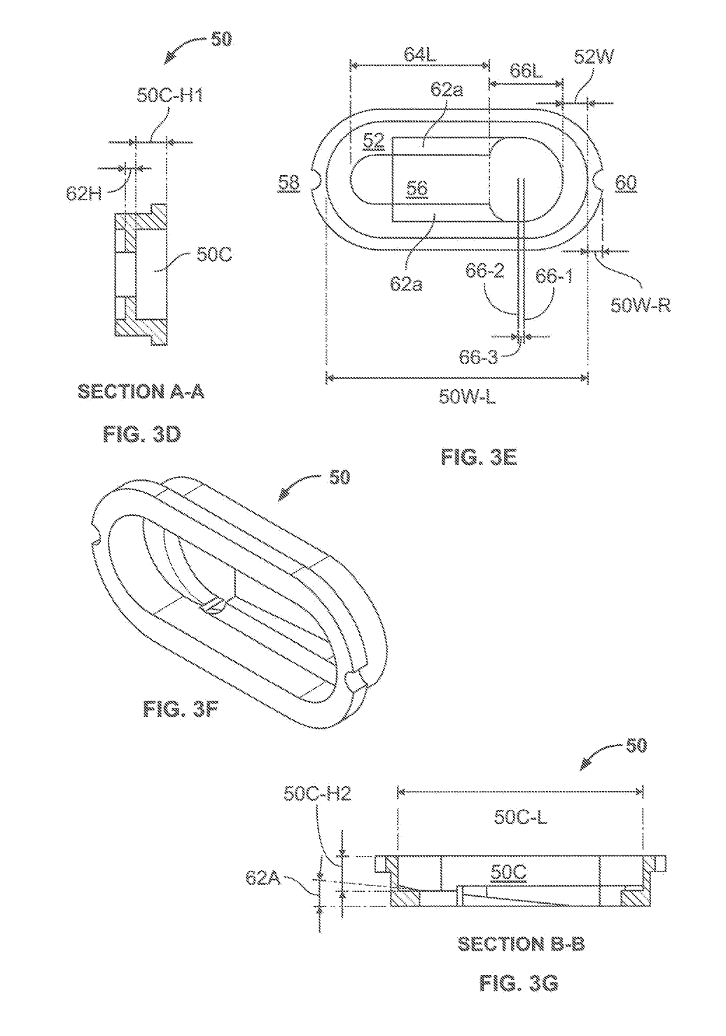

FIGS. 3A to 3F collectively illustrate the second attachment means 50, which comprises a body having an oval shape with a top 52 and bottom 54, and with an elongated opening 56 in the middle having a proximal 58 and distal end 60 said opening 56 defined by a rim 62 having a length, width, and height, a topside, and bottomside extending inwardly from the top of said body 52 providing for said attachment element 90 (e.g., screw & nut) to adjustably pass therethrough.

The first region 64 of said rim 62 having a length, width, and height being substantially elliptical in shape beginning near the proximal end 58 (front of the mouth) of said top 52 of said body. The second region 66 positioned near said distal end (rear of the mouth) 60 of said rim 62, is substantially circular, and intersects said first region 64; and wherein a section of the topside of the rim 62 comprising a positioning means 62A for receiving said attachment element 90, said rim 62 having a varying height 62H forming a declined plane 62A beginning near the proximal end 58 and terminating at the intersection of the second region 66 with an angle of eight (8) degrees, wherein the height 62H is no less than 0.020 inches, as illustrated via FIG. 3D via Section A-A.

As illustrated in FIG. 3A, the attachment means 50 has an overall length of 0.540 inches, and overall width of 0.280 inches, see 50L, 50W respectively. The elongated opening 56 has an overall length of 0.376 inches (0.234+0.142), wherein the first region 64 has a length 64L and width 64W of 0.234 and 0.102 inches respectively, whereas the second region 66 has a length 66L and width 66W of 0.142 and 0.172 inches respectively. The wall 50W between the top 52 and bottom 54 has an overall outside dimensional length 50W-L of 0.480 inches, a width 50W-W of 0.240 inches, while being recessed 50W-R from the periphery of 0.030 inches. The formation of second region 66 has a first centerline 66-1 having a radius of 0.172 nearest distal end 60, and a second centerline 66-2 radius of 0.047 inches a mere 0.021 inches 66-3 closer to the proximal end 58 than said first centerline.

Moreover, the wall 50W has a height 50W-H of 0.070 inches, creating an overall height 50H of attachment means 50 of 0.100 inches, while forming a cavity 50C, as illustrated in FIG. 3G via Section B-B, having a length 50C-L of 0.460 inches from proximal to distal end, a width 50C-W of 0.180 inches, a first height of 50C-H1 of 0.060 inches for the entire first region 64, and a second height of 50C-H2 of 0.070 inches for part of the second region 66 and part of the first region 64, and leaving the base 54 as having a width 54W of 0.040 inches around the periphery. The top 52 having a width 52W on the proximal and distal ends of 0.054 inches. The difference in height between 50C-H1 to 50C-H2 is illustrated in FIG. 3A, and Section B-B in FIG. 3G, wherein 50C-H1 is shown as hatched area, the increased depth/height of 0.010 inches provides for the positive locking mechanism feature of the means 10 receiving means 90 and providing for the rotation there between until means 90 is seated perpendicular to means 10. See FIGS. 4A to 4D.

The system further includes an attachment element 90 (e.g., screw & nut) as illustrated in FIGS. 2A to 2D collectively, wherein attachment element 90 comprises means for connecting said first attachment means 10, and connecting said second attachment means 50 cooperatively together.

The attachment element 90 comprises a male 92 and a female 94 member; said male member 92 having a head 92H, a neck 92N, a waist 92W, and a tail 92T portion; said head 92H and waist 92W portion each having a diameter both of which are larger than the diameter of said neck 92N and tail 92T portions. The diameter 92H-D of the head 92H is 0.167 inches, and the waist 92W-D is 0.160 inches. The head 92H further having a height 92H-H of 0.038 inches, a top 92H-T, a bottom 92H-B, and a side 92H-S, wherein the top 92H-T has a diameter less than the bottom 92H-B, and a rounded edge 92H-R (0.026 inch rad) connecting the top and side surfaces 92H-T, 92H-S of the head 92H.

The head portion 92H being configured and dimensioned to pass through said second region 66 but not said first region 64, wherein said head 92H can only pass through the second region 66 at an of angle of eight (8) degrees+/−3 degree. It should be noted, that although this embodiment incorporates a eight (8) degree angle, it is essential that whatever the angle, it should not be an angle that permits separation of means 10 and 50 when they are parallel to each other. Moreover, based on positioning of the means 10, 50 when installed on the respective member MM1, MM2, there is only so much tilting that would be permissible or allowable in a practical setting.

The neck portion 92N configured and dimensioned to pass through both regions 64, 66, and to slide freely between said proximal and distal end 58,60 within both regions 64, 66. The neck 92 92N having a height 92N-H of 0.070 inches and a diameter 92N-D of 0.099 inches.

The waist portion 92W being configured and dimensioned to not pass through said second region 66, nor said first region 64. The waist 92W having a height 92W-H of 0.046 inches, and a diameter 92W-D of 0.160 inches, wherein the intersection of waist's 92W sides, and top and bottom have a 0.005×45 degree type angle 92W-A.

The tail portion 92T having a height, and diameter, with a threaded means 92T-M about the sides configured and dimensioned to mate with said female member 94. The height of the tail 92T-H being 0.100 inches, and a diameter less than 0.099 inches of the neck 92N-D. The threaded means 92T-M being 3-56 UNF-2A, and having an undercut 92T-U of 0.020 inches.

The female member 94 comprising a body having a diameter 94D, height 94H, top, bottom, and sides, and orifice 96 in the center of said body configured and dimensioned to mate with said tail portion 92T. The diameter 94D having a dimension of 0.158 inches+/−0.002 inches, and the height 94H having a dimension of 0.066 inches.

The sides 94S defining an outer perimeter wherein the front 94S-F, and rear 94S-R sides each have a radius 94S-F-R, 94S-R-R wherein the rear perimeter has a radius 94S-R-R greater than the front 94S-F-R, wherein the front radius is 0.020 inches, and rear radius is 0.079 inches, wherein the orientation has the larger radius 94S-R-R facing the rear of the mouth because the curved portion is facing towards the rear of the mouth.

The rear portion 94S-R being configured to mate with the curved end of the chamber 10C of said first attachment means 10; and the sides 94S-F, 94S-R being configured to slide within the chamber 10C but prohibit rotational movement of said female member 94 within the chamber 10C; wherein said diameter 94D is greater than the narrow portion of the elongated opening of the first attachment means 10. The orifice 96 having threads 3-56 UNC-2B thru to mate with tail portion 92T having 3-56 UNF-2A.

FIGS. 4A to 4D illustrates a profile of a custom fitted and manufactured oral appliance that is constructed of a material capable of withstanding the physical and chemical stresses exerted from placement and usage in the mouth while capable of customization to the user's specific anatomical features, e.g., flexite.

In particular FIG. 4A illustrates the system 1 wherein the attachment element 90, is attached to the first attachment means 10, and is positioned at the critical angle of insertion into the second attachment means 50. Once the attachment means 10, 50 are parallel as illustrated in FIG. 4B, they are free to glide to and from, but are not able to detach when deployed in the users mouth due to the positive locking mechanism of the present invention. FIG. 4C illustrates the first attachment means 10 in the desired position on the upper mouthpiece MM1, wherein the first attachment means 10 are installed parallel to one another 10a, 10b near the curved end of the upper mouthpiece MM1.

It should be noted, the system is designed to be surface mounted and for use with molded members MM1, MM2 respectfully, although the invention does not extend to the molded members, the present invention in practice is for use with MM1 and MM2. It is essential for the means 10 to be surface mounted so as to ensure element 90 can be adjusted by the patient and/or medical technician to fine tune the system 1, that is, for the element 90 can slide back and forth in the track and be retightened once in position relative to the means 10. Moreover, it is essential for means 50 to be surface mounted as well so as to permit the free movement of element 90 when engaged with means 50.

Likewise, the second attachment elements 50a,b are positioned on the lower mouthpiece MM2, see FIG. 4D, and are configured and dimensioned to mate with the first attachment elements 10a,b shown in FIG. 4C. Again, in order for the first and second attachment means 10a,b, 50a,b to engage, the attachment means 10a,b, 50a,b the first attachment element 10a,b must be at the critical angle+/−set forth above and then tilted to be perpendicular to the plane of the second attachment elements MM2 to positively lock together, and prevent separation while the first and second attachment means 10a,b, 50a,b remain substantially parallel to each other.

1. An attachment system for use with an intra-oral dental appliance to be inserted into the oral cavity of a human for treatment of sleep apnea, snoring, bruxism, and temporo-mandibular joint disorder, said attachment system comprising:
a first attachment means securable to a bilateral under surface conforming to the clinical occlusal plane of a human, and to the maxillary dentition of a first molded member;
a second attachment means securable to a bilateral upper surface conforming to the clinical occlusal plane of a human, and to the mandibular dentition of a second molded member;
an attachment element comprising means for connecting said first attachment means, and connecting said second attachment means cooperatively together;
wherein said first attachment means comprises a body having an oval shape with a top and a base and an elongated opening in the middle for said attachment element to adjustably pass therethrough;
wherein said top includes a flange extending inwardly, and has adjustment indicators for use with said attachment element;
wherein said base includes a flange extending outwardly for securing the first attachment means to the first molded member;
said body having a wall between the top and base and runs from the length of a first long side, around a first curve to a second long side running the length thereof forming a chamber;
wherein said second attachment means comprises a body having an oval shape with a top and bottom, and with an elongated opening in the middle having a proximal and distal end said opening defined by a rim having a length, width, and height, a topside, and bottomside extending inwardly from the top of said body providing for said attachment element to adjustably pass therethrough;
a first region of said rim having a length, width, and height being substantially elliptical in shape beginning near the proximal end of said top of said body;
a second region positioned near said distal end of said rim, is substantially circular, and intersects said first region; and
wherein a section of the topside of the rim comprising a positioning means for receiving said attachment element, said rim having a varying height forming a declined plane beginning near the proximal end and terminating at the intersection of the second region;
wherein said attachment element comprises a male and a female member;
said male member having a head, a neck, a waist, and a tail portion; said head and waist portion each having a diameter both of which are larger than the diameter of said neck and tail portions; the head further having a height, a top, a bottom, and a side, wherein the top has a diameter less than the bottom, and a rounded edge having a 0.026 inch rad connecting the top and side surfaces of the head;
said head portion being configured and dimensioned to pass through said second region but not said first region, wherein said head can only pass through said second region at an of angle of greater than eight (8) degrees+/−3 degrees;
said neck portion configured and dimensioned to pass through both regions, and to slide freely between said proximal and distal end within both regions;
said waist portion being configured and dimensioned to not pass through said second region, nor said first region;
said tail portion having a height, and diameter, with a threaded means about the sides configured and dimensioned to mate with said female member;
said female member comprising a body having a diameter, height, top, bottom, and sides, and orifice in the center of said body configured and dimensioned to mate with said tail portion; the sides defining an outer perimeter wherein the front, and rear sides each have a radius, wherein the rear perimeter has a radius greater than the front; the rear portion begin configured to mate with the curved end of the chamber of said first attachment means; and the sides being configured to slide within the chamber but prohibit rotational movement of said female member within the chamber; wherein said diameter is greater than the narrow portion of the elongated opening of the first attachment means.

2-4. (canceled)

5. An attachment system for use with an intra-oral dental appliance to be inserted into the oral cavity of a human for treatment of sleep apnea, snoring, bruxism, and temporo-mandibular joint disorder, said attachment system comprising:
a first attachment means securable to a bilateral under surface conforming to the clinical occlusal plane of a human, and to the maxillary dentition of a first molded member; wherein said first attachment means comprises a body having an oval shape with a top and base and an elongated opening in the middle for an attachment element to adjustably pass therethrough;
a second attachment means securable to a bilateral upper surface conforming to the clinical occlusal plane of a human, and to the mandibular dentition of a second molded member;
said attachment element comprising means for connecting said first attachment means, and connecting said second attachment means cooperatively together.

6. An attachment system as in claim 5, wherein said top includes a flange extending inwardly, and has adjustment indicators for use with said attachment element.

7. An attachment system as in claim 5, wherein said base includes a flange extending outwardly for securing the first attachment means to the first molded member.

8. An attachment system as in claim 5, wherein said body having a wall between the top and bottom and runs from the length of a first long side, around a first curve to a second long side running the length thereof forming a chamber.

9. An attachment system as in claim 5, wherein said second attachment means comprises a body having an oval shape with a top and bottom, and with an elongated opening in the middle having a proximal and distal end said opening defined by a rim having a length, width, and height, a topside, and bottomside extending inwardly from the top of said body providing for said attachment element to adjustably pass therethrough.

10. An attachment system as in claim 9, wherein a first region of said rim having a length, width, and height being substantially elliptical in shape beginning near the proximal end of said top of said body.

11. (canceled)

12. An attachment system as in claim 9, wherein a second region positioned near said distal end of said rim, is substantially circular, and intersects said first region.

13. An attachment system as in claim 9, wherein a section of the topside of the rim comprising a positioning means for receiving said attachment element, said rim having a varying height forming a declined plane beginning near the proximal end and terminating at the intersection of the second region.

14. An attachment system as in claim 5, wherein said attachment element comprises a male and a female member.

15. An attachment system as in claim 14, wherein said male member having a head, a neck, a waist, and a tail portion; said head and waist portion each having a diameter both of which are larger than the diameter of said neck and tail portions; the head further having a height, a top, a bottom, and a side, wherein the top has a diameter less than the bottom, and a rounded edge having a 0.026 inch rad connecting the top and side surfaces of the head;
said head portion being configured and dimensioned to pass through said second region but not said first region, wherein said head can only pass through said second region at an of angle of greater than eight (8) degrees+/−3 degrees;
said neck portion configured and dimensioned to pass through both regions, and to slide freely between said proximal and distal end within both regions;
said waist portion being configured and dimensioned to not pass through said second region, nor said first region;
said tail portion having a height, and diameter, with a threaded means about the sides configured and dimensioned to mate with said female member.

16. An attachment system as in claim 14, wherein said female member comprising a body having a diameter, height, top, bottom, and sides, and orifice in the center of said body configured and dimensioned to mate with said tail portion; the sides defining an outer perimeter wherein the front, and rear sides each have a radius, wherein the rear perimeter has a radius greater than the front; the rear portion begin configured to mate with the curved end of the chamber of said first attachment means; and the sides being configured to slide within the chamber but prohibit rotational movement of said female member within the chamber; wherein said diameter is greater than the narrow portion of the elongated opening of the first attachment means.
